(19) United States
(12) Patent Application Publication
(10) Pub. No.: US 2017/0319437 A9
(43) Pub. Date: Nov. 9, 2017

(54) GELLAN GUM CARRIER FOR A MEDICAMENT, MEANS AND METHOD

(71) Applicants: VADIM ZELIKMAN, RISHON LEZION (IL); ZAREMA ZELIKMAN, RISHON LEZION (IL)

(72) Inventors: VADIM ZELIKMAN, RISHON LEZION (IL); ZAREMA ZELIKMAN, RISHON LEZION (IL); OLEG ZUKOV, ZORAN (IL)

(73) Assignees: VADIM ZELIKMAN, RISHON LEZION (IL); ZAREMA ZELIKMAN, RISHON LEZION (IL)

(21) Appl. No.: 14/654,792

(22) Filed: Jun. 22, 2015

Related U.S. Application Data
(63) Continuation-in-part of application No. PCT/IL13/51049, filed on Dec. 23, 2013.
(60) Provisional application No. 61/745,614, filed on Dec. 23, 2012.
(60) Provisional application No. 62/015,457, filed on Jun. 22, 2014.

(51) Int. Cl. A61J 3/00; B65D 81/32; A61K 9/06; A61K 47/36; A61K 9/00; A61K 31/167; A61J 7/00; A61J 1/20

(52) U.S. Cl. CPC A61J 3/00; A61K 9/0056; B65D 81/32; A61K 31/167; A61K 9/06; A61K 47/36; A61J 1/2093; A61J 7/0007

(57) ABSTRACT

The present invention provides composition, apparatus, kit and method for compounding an orally administered dosage form of at least one medicament comprising a container for mixing a crushed medicament with a gel-based carrier. The container comprises at least one chamber and at least one membrane as a separating element. The container comprises a flexible membrane, when removed or partially broken, provides a mixture of a defined dosage form of said medicament comprising the crushed medicament and said gel-based carrier. The membrane is further configured to prevent contamination, overdosing and non sanctioned abuse of active ingredient of said medicament.

### CROSS REFERENCE

This application is a national phase of PCT Application Number PCT/IL2013/051049, filed on Dec. 23, 2013, which claims priority from provisional application No. 61/745,614, filed on Dec. 23, 2012 and provisional application No. 62/015,457, filed on Jun. 22, 2014. All of these applications are hereby incorporated by reference in their entirety.

### FIELD OF INVENTION

The present invention pertains to compositions comprising a gel material such as Gellan gum, Pregellatinized starch or Modified Starch adapted as a carrier for a pharmaceutical drug or the like. The invention further relates to an apparatus, kit and method for compounding an orally administered dosage form of a medicament.

### BACKGROUND OF THE INVENTION

In the medicinal field one or more coatings is desired for drug administration forms such as tablets, pills caplets and the like in order to obtain one or more of gloss, better appearance, identification, mouth feel, stability, color, swallowability, improved taste and the like.

Variety of coating material are used for medicaments especially gel materials as gellan gum.

US patent application No. 2008299199 describes a dosage form which before oral ingestion the particulate material is subjected to an aqueous medium, whereby it is converted to a semi-solid form by swelling or geling of one or more of the components, especially of a gellan gum, of the particulate matter. The invention also relates to a vehicle for oral administration of one or more active substances, the vehicle comprising a gellan gum arranged in a configuration allowing optimal water diffusion so that upon addition of a predetermined amount of an aqueous medium, without the necessity of applying shear forces or other mixing forces, within a time period of 5 minutes or less swells and/or gels and the texture of the swelled vehicle being similar to that of a soft pudding and having a viscosity of at least about 10,000 cps.

EP patent No. 0662320 further discloses a dry gel composition containing 40 wt. % or less of an orally administrable medicine, 3 wt. % or more of a geling agent and 5 wt. When mixed with a given amount of water, the composition gives a homogeneous aqueous gel composition having a viscosity of around 100 to 500 cP and preferably a thixotropy. The aqueous gel composition can be readily swallowed even by the aged having a reduced swallowing function without being aspirated into the trachea, so that it is useful for the pharmacotherapy of the aged.

US patent No. 2004033261 describes a film coated tablet wherein the tablet is film coated with a gellan gum coating composition containing gellan gum, a plasticizer, and a disintegration aid. Optionally a slip enhancer is added to the composition. A method for coating a tablet with the gellan gum composition wherein the composition is applied as a solution. A method of administering the gellan gum film coated tablet as a pharmaceutical.

The above prior art describes the formation of an aqueous gel composition and further the convenient of the use and preparation but they don't focused on the dosage form protection.

There is a desire for a product which provides enhanced tablet coating properties. The industry has recognized the need for an improved tablet coating which would provide increased gloss, better mouth feel at coating quantities at lower levels than conventionally accepted methods. The process of preparing such an improved tablet coating economically and efficiently continues to be of interest.

It therefore remains a long felt and unmet need to provide novel means and methods for an effectively composition for drug administration forms in a more effective and adjustable manner.

### SUMMARY OF THE INVENTION

It is thus one object of the present invention to disclose an apparatus for compounding an orally administered dosage form of at least one medicament comprising: a container for mixing a crushed medicament with a gel-based carrier; the container comprises at least one chamber and at least one membrane as a separating element; wherein the membrane, when removed, provides a mixture of a defined dosage form of the medicament comprising the crushed medicament and the gel-based carrier; the membrane is further configured to prevent contamination, overdosing and non sanctioned abuse of active ingredient of the medicament.

It is a further object of the present invention to disclose the apparatus as defined above, wherein the container comprising at least one first chamber for accommodating a solution and at least one second chamber for accommodating a gel-based carrier; the at least one first chamber and at least one second chamber are connected with each other via a membrane for separating the first and second chambers; the membrane, when removed, provides a mixture of a defined dosage form of the medicament comprising the crushed medicament and the gel-based carrier, the first chamber and second chamber have a surface tension allowing the user to extract the mixture by pressing at least one chamber.

It is a further object of the present invention to disclose the apparatus as defined in any of the above, wherein at least one of the following hold true:
a. the membrane comprising a reversibly sealed orifice such that when opened, provides a mixture of a defined dosage form of the medicament comprising the crushed medicament and the gel-based carrier; the reversibly sealed orifice is opened by manual action or a manual or electronic button;
b. the membrane is partially broken by rotating the first chamber at least a half-turn therefore, providing a defined dosage form of the medicament comprising the crushed medicament and the gel-based carrier, the membrane is further configured to prevent contamination, overdosing and non sanctioned abuse of active ingredient of the medicament;
c. additionally comprising a collecting device selected from the group consisting of a spoon, a cup and a combination thereof; and
d. additionally comprising a crushing apparatus for crushing a medicament.

It is a further object of the present invention to disclose the apparatus as defined in any of the above, wherein at least one of the following holds true:
a. the membrane is a composed of a flexible material selected from the group consisting of polymer, cellulose, metal, metal alloys, metal oxides and any combination thereof;
b. the membrane is partially broken by tapping, knocking, raping or hitting the upper chamber thereby forming an aperture within a portion of the membrane;
c. the dosage form selected from the group consisting of: aliquot, tablet, caplet, capsule, pill, bolus, particle, micronized particle, particulate, pellet, core, powder, granule, granulate, small mass, seed, specks, spheres, crystals, beads, agglomerates, nanoparticles or any combination thereof;
d. at least one chamber is an upper chamber comprising at least one mold adjusted to a structure of the dosage form of at least one medicament; and
e. at least one first chamber is adapted for accommodating the gel-based carrier whilst the second chamber is adapted for accommodating water and salt;

It is a further object of the present invention to disclose the apparatus as defined in any of the above, wherein at least one of the following holds true:
a. the salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
b. the container is suitable for customizing a tailor made prescription to fit individual requirements in a dosage form that insures efficacy, swallowability, safety and compliance;
c. the mold has a cavity shape selected to accommodate a dosage form selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; the mold is a replaceable element which can be removed and/or replaced;
d. the gel-based carrier is selected from the group consisting of gellen gum, pregellatinized starch, modified starch, Pionil 1500, GENU GEL SWG-J, Primojel, GENU pectin, guaiacol, L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof; and
e. the container additionally comprising a syringe or a needle attached to at least one edge of the container for dispensing the mixture.

It is a further object of the present invention to disclose a kit for oral administration of at least one medicament comprising:
a. an apparatus for compounding an orally administered dosage form of a medicament comprising:
   i. a container for mixing a crushed medicament with a carrier the container comprising at least first chamber and at least second chamber connected with each other via a membrane for separating the chambers; and,
   ii. a collecting device;
b. a crushing apparatus for crushing a medicament;
c. a medicament; and,
d. a gel-based carrier selected from the group consisting of gellan gum, pregellatinized starch, modified starch and a combination thereof;
wherein the membrane is removed or partially broken by rotating the first chamber at least a half-turn therefore, providing a mixture of defined dosage form of a defined medicament comprising the crushed medicament and the gel-based carrier, so as to prevent contamination, overdosing and non sanctioned abuse of active ingredient of the medicament.

It is a further object of the present invention to disclose the kit as defined in any of the above, wherein the membrane, when removed, the content of each of the chamber is mixed thereby, forming a mixture of a defined dosage form of a defined medicament comprising the crushed medicament and the gel-based; the first chamber and second chamber have a surface tension allowing the user to extract the mixture by pressing at least one chamber.

It is a further object of the present invention to disclose the kit as defined in any of the above, wherein at least one of the following holds true:
a. the dosage form is selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof;
b. at least one chamber comprising at least one mold adjusted to a structure of the dosage form of at least one medicament;
c. the membrane is partially broken by tapping, knocking, raping or hitting the first chamber thereby forming an aperture within a portion of the membrane;
d. the mold has a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; the mold is a replaceable element which can be removed and/or replaced; and
e. the collecting device is selected from the group consisting of spoon, cup and any combination thereof.

It is a further object of the present invention to disclose the kit as defined in any of the above, wherein least one of the following holds true:
a. at least one chamber is adjusted to the dosage form of the medicament preventing contamination, overdosing and non sanctioned abuse of active ingredients of the medicament;
b. the kit is suitable for customizing a tailor made prescription to fit individual requirements in a dosage form that insures efficacy, swallowability, safety and compliance;
c. the second chamber is adapted for accommodating water and salt;
d. the salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations; and
e. the gel-based carrier is further selected from the group consisting of Pionil 1500, GENU GEL SWG-J, Primojel, GENU pectin, guaiacol, L-HPC (LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof.

It is a further object of the present invention to disclose a composition solid dosage form composition for oral administration prepared by a process comprising steps of:
a. providing a gel-based carrier adapted for mixing with orally administrated medicament; the gel-based carrier is selected from the group consisting of: gellan gum, pregellatinized starch, modified starch and a combination thereof;
b. providing a crushed or powdered dosage form of a medicament;
c. providing an apparatus for compounding an orally administered dosage form of a medicament comprising:
   i. a container for mixing a crushed medicament with a gel-based carrier; the container comprising at least one first chamber and at least one second chamber connected with each other via a membrane for separating the first chamber and the second chamber;
   ii. a crushing apparatus for crushing a medicament; and,
   iii. a collecting device;
d. admixing the content of the first chamber comprising the carrier and the second chamber comprising water under shear to prepare a solid composition;
   wherein the step of admixing the content is by removing the membrane from the container thereby, providing a mixture of a defined dosage form of the medicament comprising the crushed medicament and the gel-based carrier; further wherein the first chamber and second chamber have a surface tension allowing the user to extract the mixture by pressing at least one chamber.

It is a further object of the present invention to disclose the composition as defined in any of the above, wherein the step of admixing the content is by additionally rotating the first chamber at least a half-turn membrane such that the membrane is partially broken therefore, providing a mixture of defined dosage form of a defined medicament comprising the crushed medicament and the gel-based carrier, so as to prevent contamination, overdosing and non sanctioned abuse of active ingredient of the medicament.

It is a further object of the present invention to disclose the composition as defined in any of the above, wherein at least one of the following holds true:
a. the solid composition is useful for improving swallowing form, smell and taste of the medicament it comprises; and
b. the membrane is partially broken by tapping, knocking, raping or hitting at least one chamber thereby forming an aperture within a portion of the membrane.

It is a further object of the present invention to disclose the composition as defined in any of the above, wherein at least one of the following holds true:
a. further comprising additives as a palatability improving agents;
b. the solid composition further comprising an effective amount of a plasticizer, a disintegration aid, or an effective amount of a slip enhancer;
c. the composition further comprises binders selected from the group consisting of: hydroxypropyl cellulose, hydroxypropyl methylcellulose and polyvinylpyrrolidone; and
d. the composition further comprises ingredients selected from the group consisting of: flavor(s), sweetener(s), mint(s), fragrance(s), active ingredient(s) and mixtures thereof.

It is a further object of the present invention to disclose the composition as defined in any of the above, wherein at least one of the following holds true:
a. the dosage form is selected from the group consisting of: aliquot, tablet, caplet, capsule, pill, bolus, particle, micronized particle, particulate, pellet, core, powder, granule, granulate, small mass, seed, specks, spheres, crystals, beads, agglomerates, nanoparticles or any combination thereof;
b. at least one chamber comprising at least one mold adjusted to a structure of the dosage form of at least one medicament; the mold is with a cavity shape to accommodate a dosage form selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; the mold is a replaceable element which can be removed and/or replaced; and
c. the collecting device is selected from the group consisting of: spoon, cup or the like.

It is a further object of the present invention to disclose the composition as defined in any of the above, wherein at least one of the following holds true:
a. the gellan gum is provided in a weight of about 0.025% to about 30% weight percent of the total dosage form weight; and
b. the gellan gum is provided in a weight of preferably about 0.05% to about 5% weight percent of the total dosage form weight.

It is a further object of the present invention to disclose the composition as defined in any of the above, wherein at least one of the following holds true:
a. at least one chamber comprising water with a salt selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
b. the sweeteners is selected from the group consisting of corn syrup solids, sucrose, aspartame, neotame, maltilol, maltilol syrup, neosorb, xylitol, acesulfame, Sweet Am, fructose, brown sugar, Stevia, saccharin and a combination thereof;
c. the flavors is selected from the group consisting of cherry, mint, spearmint, peppermint, wintergreen, banana, coconut, wild cherry, grape, tooti fruiti, cinnamon, strawberry, orange, root beer, bubble gum, chocolate and any combination thereof; and
d. the gel-based carrier is selected from the group consisting of Pionil 1500, GENU GEL SWG-J; Primojel, GENU pectin, guaiacol; L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof.

It is a further object of the present invention to disclose a method for preparing a composition for oral administration of at least one medicament, comprising steps of:
a. providing a kit for oral administration of a medicament comprising:
   i. a container for mixing a crushed medicament with a gel-based carrier comprising at least one first chamber and at least one second chamber connected with each other via a membrane for separating the chambers a crushing apparatus for crushing a medicament; and,
   ii. a collecting device;
   iii. a medicament; and
   iv. a gel-based carrier selected from the group consisting of gellan gum, pregellatinized starch, modified starch and a combination thereof;
b. crushing the medicament using a crushing apparatus;
c. adjusting at least one chamber to the dosage form of the medicament;
d. applying the carrier to the first chamber of the container whilst the second chamber comprises water and salt solution;
e. removing or partially breaking the membrane of the container; and
f. admixing the first chamber and second chamber comprising the gel-based carrier, medicament and water under shear to prepare a solid composition.

It is a further object of the present invention to disclose the method as defined in any of the above, wherein at least one of the following holds true:
a. the method additionally comprising the step of providing the gellan gum as an enteric coating of the medicament; the enteric coating is adjusted to the dosage form of the medicament in order to prevent contamination, overdosing and non sanctioned abuse of active ingredients of the medicament;
b. the method further comprising the step of adding a salt solution to the chamber comprising water; the salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
c. the method further comprising the step of adding an effective amount of a plasticizer, a disintegration aid, or an effective amount of a slip enhancer;
d. the method additionally comprising step of providing the gellan gum in a weight of preferably from about 0.05% to about 5% weight percent of the total medicament dosage form weight; and
e. the method additionally comprising step of providing the gellan gum in a weight of about 0.025% to about 30% weight percent of the total medicament dosage form weight.

It is a further object of the present invention to disclose the method as defined in any of the above, wherein at least one of the following holds true:
a. the step of providing the membrane comprising a reversibly sealed orifice; and

b. the method additionally comprising step of opening the membrane's orifice by using a manual action or a manual or electronic button; It is a further object of the present invention to disclose the method as defined in any of the above, wherein at least one of the following holds true:
a. the step of breaking partially the membrane is further by tapping, knocking, raping or hitting the first chamber thereby forming an aperture within a portion of the membrane;
b. the step of adjusting at least one chamber to the dosage form of the medicament using a mold having a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof. and
c. the step of providing the container comprising a syringe or a needle attached to at least one edge of the container for dispensing the mixture.

### BRIEF DESCRIPTION OF THE INVENTION

In order to understand the invention and to see how it may be implemented in practice, a few preferred embodiments will now be described, by way of non-limiting example only, with reference to be accompanying drawings, in which:

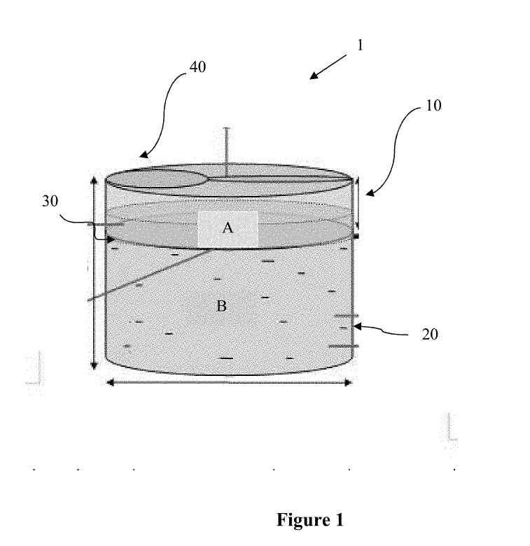

FIG. 1 presents a device having a configuration for mixing a medicament with a gel-based carrier of the present invention;

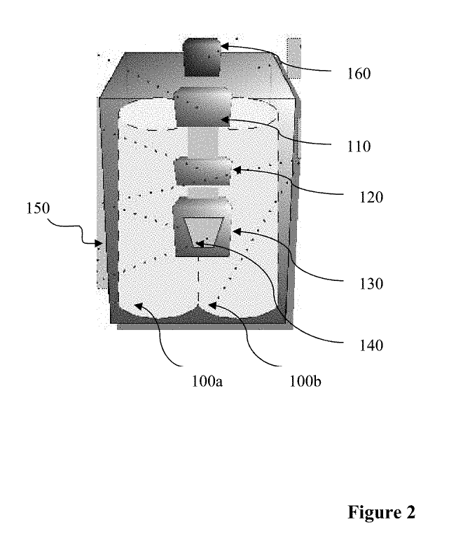

FIG. 2 presents a device having a configuration for mixing a medicament with a gel-based carrier of the present invention;

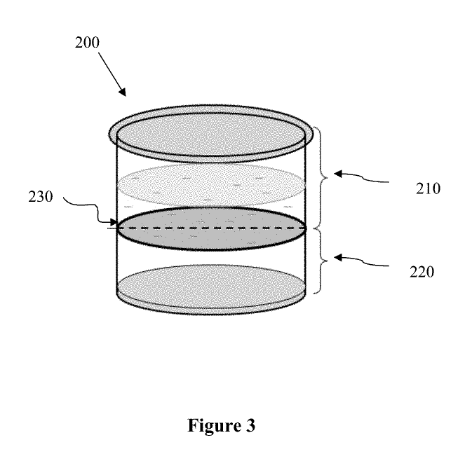

FIG. 3 presents a device having a configuration for mixing a medicament with a gel-based carrier of the present invention;

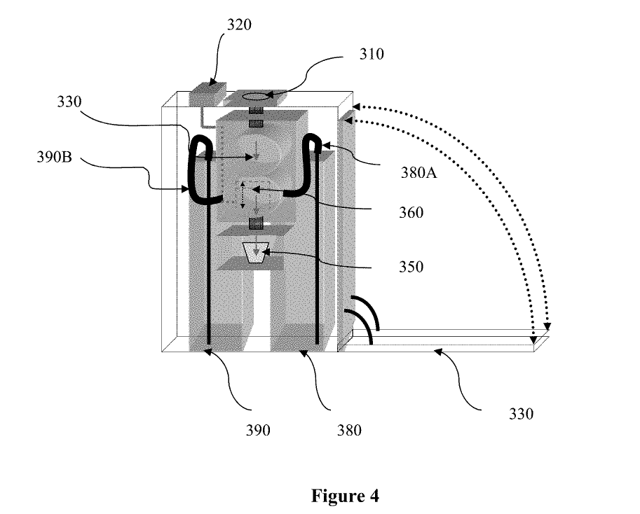

FIG. 4 presents a device having a configuration for mixing a medicament with a gel-based carrier of the present invention;

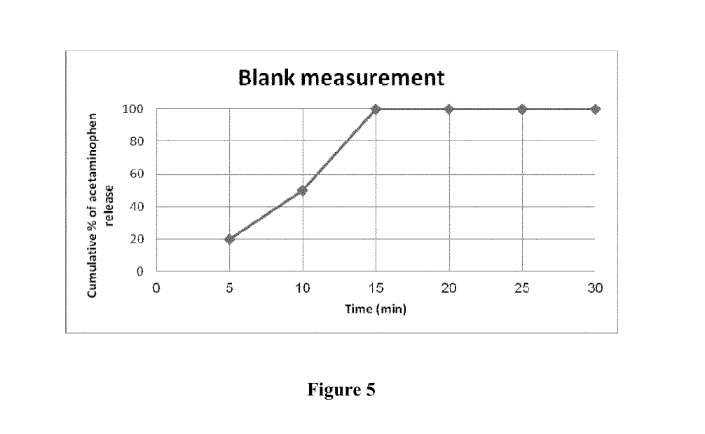

FIG. 5 presents a graph of Acetaminophen release profile of the present invention;

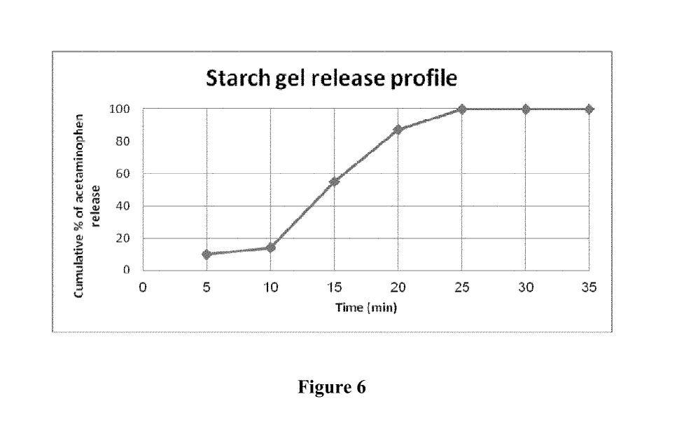

FIG. 6 presents a release profile graph of Acetaminophen incorporated with starch gel of the present invention;

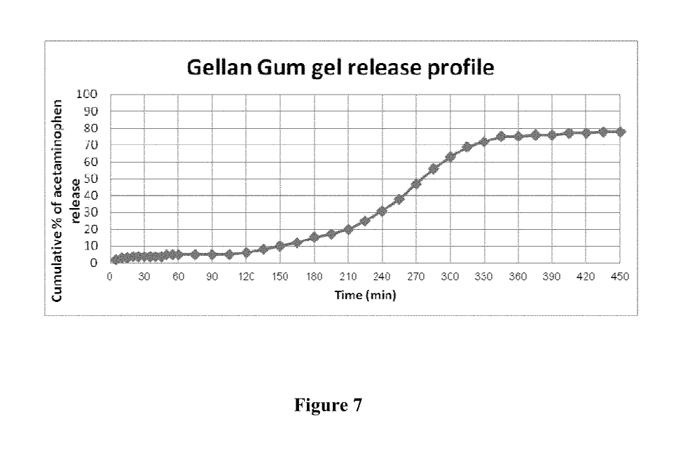

FIG. 7 presents a release profile graph of Acetaminophen incorporated with Gellan Gum Gel of the present invention;

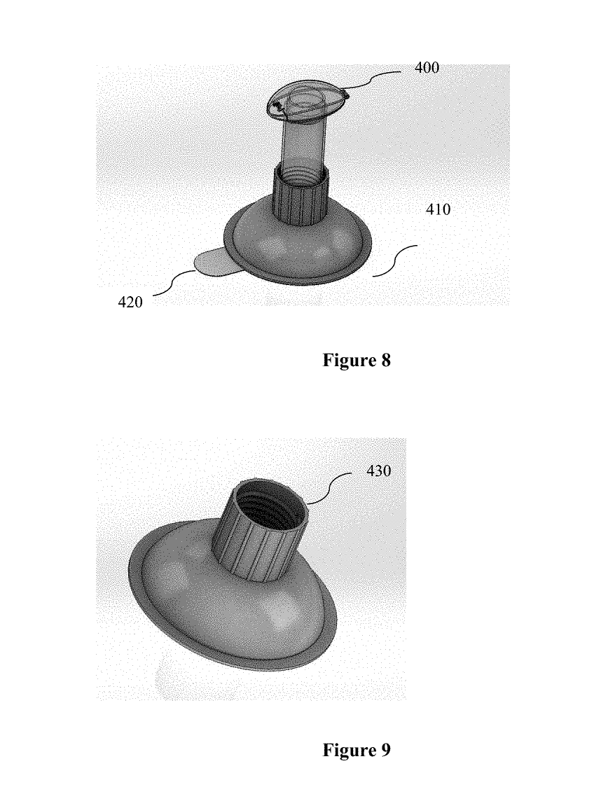

FIG. 8 presents a device of the present invention for compounding an orally administered dosage form of at least one medicament;

FIG. 9 presents the distal portion of the present invention comprising a chamber for containing a gel-based carrier;

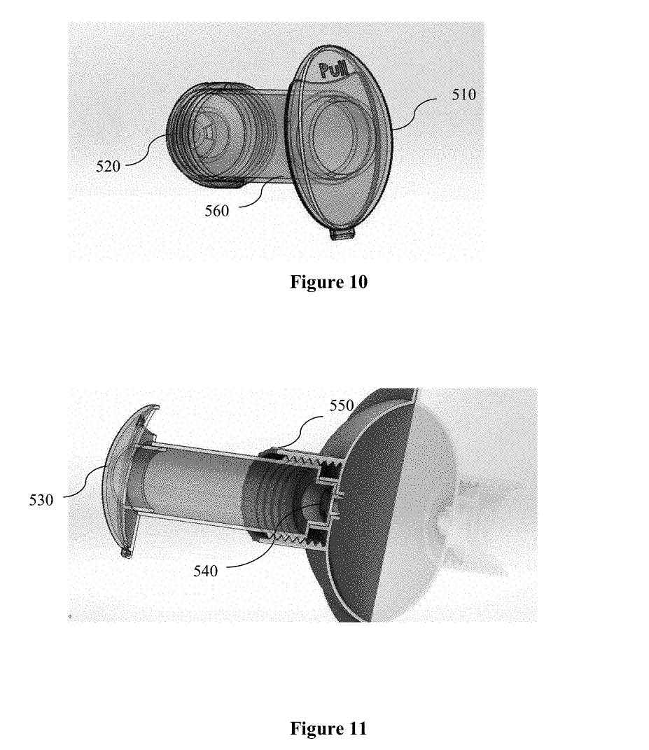

FIGS. 10 and 11 present a cross section of device of the present invention for compounding an orally administered dosage form of at least one medicament;

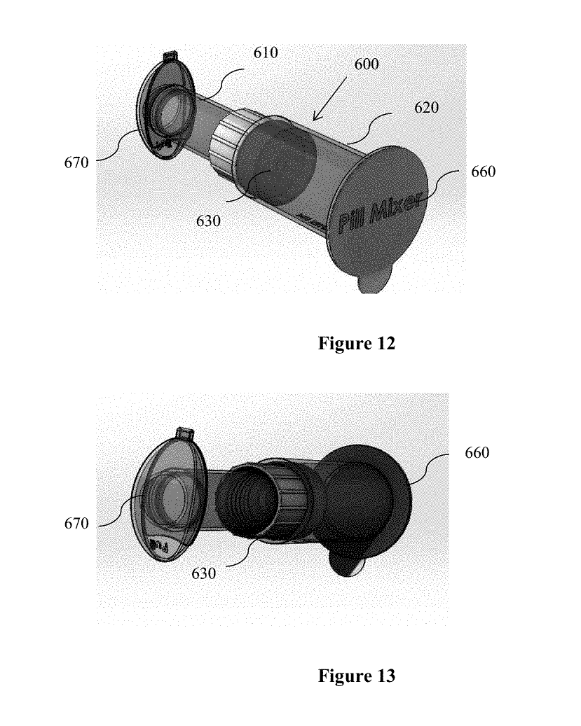

FIGS. 12 and 13 present a configuration of the present device for compounding an orally administered dosage form of at least one medicament;

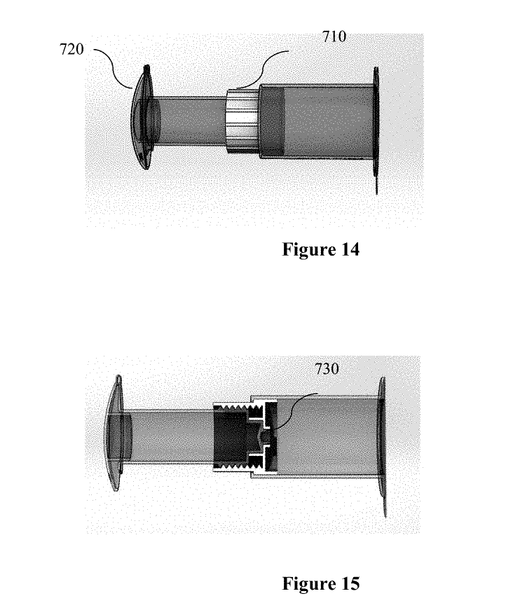

FIGS. 14 and 15 present a cross section of device of the present invention for compounding an orally administered dosage form of at least one medicament;

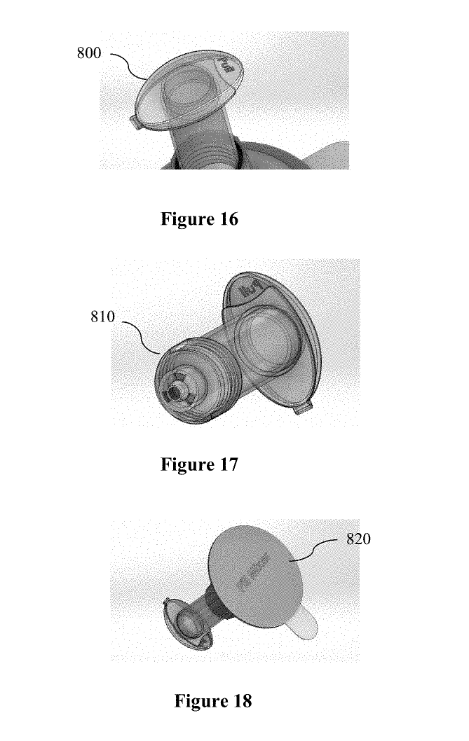

FIGS. 16-18 present a upper view of the device of the present invention for compounding an orally administered dosage form of at least one medicament; and

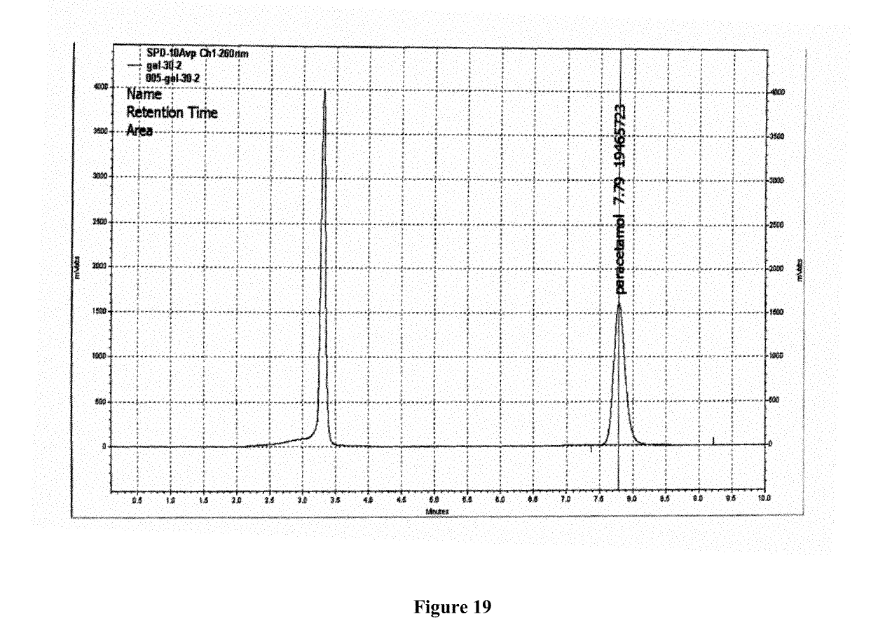

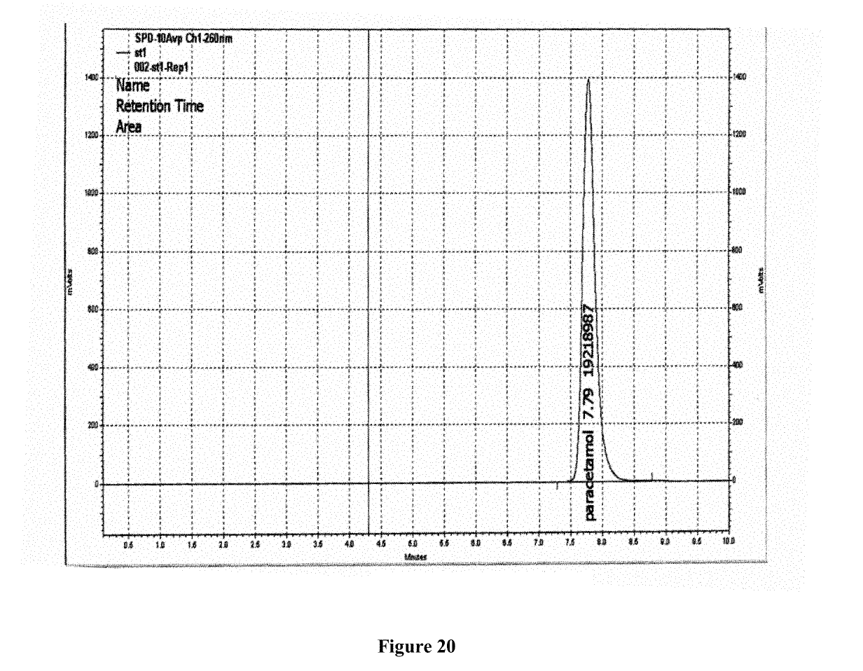

FIGS. 19 and 20 present an HPLC chromatogram of paracetamol in paracetamol gel, of the present invention.

### DETAILED DESCRIPTION OF THE INVENTION

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of the claims.

As employed herein, the term “Gellan Gum” includes gellan gum and/or compositions of gellan gum. The composition may further comprise polymers. The Gellan gum is a water-soluble polysaccharide solution which is used without limitation, as a thickener, emulsifier, and stabilizer. The gellan gum composition may further be prepared in tap water, deionized water, or other aqueous media containing salts or other components such as colorants, flavoring agents, active ingredients such as deodorants, foods, preservatives, etc.

As employed herein, the term “Dosage Form” includes without limitation, aliquot, tablet, caplet, capsule, pill, bolus, particle, micronized particle, particulate, pellet, core, powder, granule, granulate, small mass, seed, specks, spheres, crystals, beads, agglomerates, nanoparticles, mixtures thereof and the like. Furthermore the preferred dosage form will be in a form sufficiently stable physically and chemically to be effectively mixed in a system which involves some movement of the dosage form. Virtually any tablet, placebo, the latter typically lactose or sugar or mixtures thereof and the like. Furthermore the preferred dosage form may be in a liquid form.

The invention relates to an apparatus for preparing a swallowable pharmaceutical composition for oral administration comprising at least one pharmaceutically active compound in an effective amount and further comprising a carrier consisting of gellan gum, pregellatinized starch, various kinds of native and modified starches, maltodextrin, dextrins, and a kit and a process for production thereof.

As used herein the term “about X” or “approximately X” or “substantially X” usually refers to a range 25% less than to 25% more than of X (X+−25%), at times X+−20%, X+−15% and preferably X+−10%.

The present invention provides an apparatus for compounding an orally administered dosage form of at least medicament comprising: a container for mixing a crushed medicament with a carrier. The container comprises at least one chamber and a separating element. The container may additionally comprise a collecting device for collecting the prepared composition. The apparatus is adapted to provide a defined dosage form of the medicament comprising the crushed medicament and carrier such as a gel-based carrier. The apparatus is further adapted to prevent contamination, overdosing and non sanctioned abuse of active ingredients. The container may comprise a separating element such as a membrane or any divider element such that the container is divided to at least two portions or chambers. The separating element may be a mobile part such that when it is removed the content of each section within the container is mixed. The membrane has a film-like structure for separating at least two fluid and/or solid materials. It may act as a separating structure or a selective barrier allowing some particles or chemicals to pass through, but not others. The membrane may comprises at least one pore having a diameter size for allowing medicament particles to transform from the first chamber to the second chamber. The membrane pores size is selected from the group consisting of microporous (dp<2 nm), mesoporous (2 nm<dp<50 nm), macroporous (dp>50 nm) and any combination thereof. The e can be neutral or charged, and particles which transport can be active or passive. The latter can be facilitated by pressure, concentration, chemical or electrical gradients of the membrane process.

The container may further comprise at least two separated chambers which may be located one upon the other or one parallel to another. The chambers are connected with each other via the separating element such as membrane or a divider for separating the chambers.

In another embodiment of the present invention, the apparatus may be use for multiple uses and applications or for one time use respectively.

In another embodiment of the present invention the apparatus may comprise a syringe or a dispensing needle assembly which may be attached or mounted to at least one chamber of the container. The apparatus may be a sterile apparatus which configured to prevent direct contact with the environment and helping to reduce the danger of contamination and infection.

In another embodiment of the present invention the apparatus may comprise in one chamber at least one active agent in a solid or liquid form and a sterile solution in the second chamber such as NaCL, Lidocain, Lignocain, Novocain, water injection6 and the like which can be mixed with the active agent when the membrane is removed or activated and further useful for injection.

Reference is now made to FIG. 1 which presents an apparatus for preparing a pharmaceutical composition for oral administration. The dispensing unit 1 is a container configured for mixing gellan gum with a crushed medicament. The apparatus is adapted for compounding an orally administered dosage form of a medicament comprising: (a) a container for mixing a crushed medicament with a gel-based carrier, comprising at least one first chamber 20 (B) and at least one second chamber 10(A) connected with each other via a separation element 30 for separating the chambers (A, B), the apparatus further comprises a crushing apparatus for crushing the medicament.

In another embodiment of the present invention, the membrane is configured, when removed or partially broken, to provide a defined dosage form of a defined medicament so as to prevent contamination, overdosing and non sanctioned abuse of active ingredients of the medicament. The first chamber 10 may comprise at least one mold adjusted to a structure of the dosage form of at least one medicament. The mold has a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof. The mold may further be a replaceable element which can be removed and/or replaced.

In another embodiment of the present invention, the lower chamber may comprise at least one mold adjusted to a structure of the dosage form of at least one medicament. The mold has a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof. The mold may further be a replaceable element which can be removed and/or replaced.

The medicament mold plate may be of metal or metal alloys. The medicament triturate molds is a cavity plate which has holes. The volume of the cavities always remains constant, but the weight of the medicament dosage form will be depended upon the nature of the material. Different medicament dosage form will have different densities and so the cavity capacity must be determined for each medicament dosage form. The mold is further calibrated.

The container is adapted for customizing tamper proof production of swallowable aliquots. The separation element is a flexible membrane which separates the two chamber such that when it is removed the content of each of the chambers is mixed. The removing of the membrane allows mixing a variety of components in order to prepare a product suitable to be consumed as a pharmaceutical/cosmoceutical/veterinary.

The chambers of the container have a surface tension allowing the user to extract the mixed composition to a built-in spoon by pressing the chamber. The composition can be further molded into a desired shape or pressed onto a dispensing unit, a build in collecting element 40 selected from the group consisting of a spoon or a cap like element.

The apparatus may further comprise an instruction leaflet which describes the manner and way for using the apparatus its elements.

Reference is now made to FIG. 2 which presents another configuration of an apparatus for preparing a pharmaceutical composition for oral administration. The apparatus is a container configured for mixing carrier with a crushed medicament. The dispenser may be adapted for multiple uses. The dispenser comprises at least two chambers 100a and 100b, which comprises a carrier such as modified starch, pregellatinized starch or gellan gum, a crushing apparatus 110 comprising the medicament, and a mechanical handle 150 which controls and allows the crushing of the medicament and transferring of the carrier from at least one of the chambers to a mixing container 120. The container also comprises the crushed medicament. The handle further allows and controls the mixing process of the carrier together with the crushed medicament. The apparatus further comprises a pressing button 160 for releasing the prepared mixture composition into a collecting device such as cup 140, located within a cubical 130 of the apparatus. The crushing device may further comprise a mold having a cavity shape adjusted to the dosage form of the medicament.

Reference is now made to FIG. 3 which presents an additional configuration of an apparatus for preparing a pharmaceutical composition for oral administration. The dispensing unit is a container 200 configured for mixing carrier with a crushed medicament. The container comprises a non removable membrane 230. The membrane is with a flexibility such that when rotating, spinning or turning the upper chamber 210 at least half-turn, the membrane is partially broken resulting a pore or an aperture having a diameter of about 0.1 mm to about 10 mm such that the content of the upper chamber 210 falls directly to the lower chamber 220. The membrane may further partially brake by tapping, knocking, raping or hitting at least one chamber thereby forming an aperture within a portion of the membrane.

The apparatus may be composed of rigid or semi-rigid material selected from a group consisting of polymers, metals, metal oxides, metal alloys, wood, cellulose and any combination thereof. The apparatus and more particularly the membrane is composed of a flexible, semi rigid material selected from the group consisting of Acrylic and modified acrylic plastics, Acrylonitrile/butadiene/styrene co-polymer, Acrylonitrile/butadiene/styrene/methyl methacrylate copolymer, Acrylonitrile/styrene copolymer, n-Alkylglutarimide/acrylic copolymers, Cellophane, Cross-linked polyacrylate copolymers, 1,4-Cyclohexylene dimethylene terephthalate, 1,4-cyclohexylene dimethylene isophthalate copolymer, Ethylene-acrylic acid copolymers, Ethylene-carbon monoxide copolymers, Ethylene-1, 4-cyclohexylene dimethylene terephthalate copolymers, Ethylene-ethyl acrylate copolymers, Ethylene-methyl acrylate copolymer resins, Ethylene/1,3-phenylene oxyethylene isophthalate/terephthalate copolymer, Ethylene-vinyl acetate copolymers, Ethylene-vinyl acetate-vinyl alcohol copolymers, Fluorocarbon resins, Hydroxyethyl cellulose film, water-insoluble, Isobutylene polymers, Isobutylene-butene copolymers, Nylon resins, Olefin polymers, Perfluorocarbon resins, Polyarylate resins, Polyaryletherketone resins, Polyarylsulfone resins, Poly-1-butene resins and butene/ethylene copolymers, Polycarbonate resins, Polyestercarbonate resins, Polyester elastomers, Polyetherimide resin, Polyethylene resins, carboxyl modified, Polyethylene, chlorinated, Polyethylene, fluorinated, Polyethylene, oxidized, Polyethylene phthalate polymers, Poly(phenyleneterephthalamide) resins, Poly(p-methylstyrene) and rubber-modified poly(p-methyl styrene), Poly(oxy-1,2-ethanediyloxycarbonyl-2,6-naphthalenediylcarbonyl) resins, Polystyrene and rubber-modified polystyrene, Polysulfide polymer-polyepoxy resins, Polysulfone resins, Poly (tetramethylene terephthalate), Polyvinyl alcohol film, Polyurethane resins, Styrene block polymers, Styrene-maleic anhydride copolymers, Styrene-methyl methacrylate copolymers, Textryls, Urea-formaldehyde resins in molded articles, Melamine-formaldehyde resins in molded articles, Vinyl chloride-ethylene copolymers, Vinyl chloride-hexene-1 copolymers, Vinyl chloride-lauryl vinyl ether copolymers, Vinyl chloride-propylene copolymers, Vinylidene chloride/methyl acrylate copolymers, Vinylidene chloride/methyl acrylate/methyl methacrylate polymers and any combination thereof. The membrane may further be partially broken by rotation of the upper chamber relative to the lower chamber or by pushing, pressing or/and clicking the upper chamber. The membrane is partially broken to enable to content of the upper chamber to be transferred to the lower chamber.

The container further comprises an upper chamber for containing water and a lower portion for accommodating the gellan gum and/or additional additives. The medicament may be located in the lower chamber with the gellan gum or added and dissolved in the water located in to the upper chamber of the container.

Reference is now made to FIG. 4 which illustrates an additional configuration of an apparatus for preparing a pharmaceutical composition for oral administration.

The apparatus comprises an opening for medicament entrance 310. The opening may further comprise a medicament identifier for medicament type identification. The medicament is transferred to a crushing apparatus 330 using a pressing button 340.

The apparatus further comprises at least one chamber 360 for mixing the crushed medicament, the gel based carrier, water and salts. The apparatus additionally comprises two separated containers 390, 380 which are connected to the mixing chamber using lines 380A, 390B for supplying a sufficient amount of gel based carrier and solutions to the mixing chamber 360. The apparatus is mechanically operated using a handle 330 which activates the apparatus. The prepared mixture is transferred to a cup 350.

The present invention further provides a kit comprising a device for mixing a crushed medicament with a carrier consisting of gellan gum, pregellatinized starch or modified starch, using water and/or salt solutions. The kit for oral administration of a medicament comprises: (a) a container for mixing a crushed medicament with a carrier comprising at least one first chamber and at least one second chamber connected with each other via a membrane for separating the chambers, (b) at least one additive, (c) a crushing apparatus for crushing a medicament, (d) a collecting device and (e) a medicament.

The kit is configured to the dosage form of the medicament to prevent contamination, overdosing and non sanctioned abuse of active ingredients.

The present invention may further comprise a kit for oral administration of a medicament comprising: (a) an apparatus for compounding an orally administered dosage form of a medicament comprising: (i) a container for mixing a crushed medicament with a carrier the container comprising a first chamber and a second chamber connected with each other via a membrane for separating the chambers; (ii) a crushing apparatus for crushing a medicament; and, (iii) a collecting device; (b) a medicament, and (c) a gel-based carrier consisting of gellan gum, pregellatinized starch or modified starch;

The kit is configured to provide a defined dosage form of a defined medicament so as to prevent contamination, overdosing and non sanctioned abuse of active ingredients.

The kit comprises a container having at least two flexible chambers whilst the first chamber may comprise a gel-based carrier, additional additives and the crushed medicament; the second chamber may comprise water and salt solution. Furthermore the carrier may be located in the lower chamber and the water and salt solution may be located in the upper chamber respectively. The medicament may be further added to the upper chamber or the lower chamber.

In another embodiment of the invention, the container comprises a flexible membrane for separating the two chambers such that when the membrane is removed the content of each of the chambers is mixed. The chambers have a surface tension allowing the user to extract the mixed composition to a built-in spoon by pressing the chamber.

In another embodiment of the invention, the membrane may be a flexible membrane such that when rotating the first chamber at least a half-turn, the membrane is partially broken therefore forming an aperture allowing the content of the first chamber to pass to the second chamber. The membrane may further partially brake by tapping, knocking, raping or hitting at least one chamber thereby forming an aperture within a portion of the membrane.

By further admixing the mixture a defined dosage form of a defined medicament is formed so as to prevent contamination, overdosing and non sanctioned abuse of active ingredient of the medicament.

In another embodiment of the invention, the kit may further comprise an instruction leaflet which describes the manner and way for using its elements and further mixing a desired medicament with a carrier.

The kit of the present invention comprises a home use or doctor's office device which, by it's specific features, provides a means of taking a tablet for example, converting it to powder (by a built-in crushing apparatus or the like), mixing it with gellan gum or another carrier, to provide a swallowable and safer bolus or aliquot, all the while protecting the active ingredient.

The kit of the present invention is with the ability to produce the customized aliquots, and furthermore is with the ability to ensure that only medically sanctioned dosages can be prepared. The present invention is suitable and designed with drug administration and anti tampering and safety standards in mind.

The chamber that comprises the medicament will be adjusted to the dosage, aliquot, tablet, caplet, capsule, pill, bolus, particle, micronized particle, particulate, pellet, core, powder, granule, granulate, small mass, seed, specks, spheres, crystals, beads, nanoparticles, agglomerates, of a specific medicament to prevent contamination, overdosing and non sanctioned abuse of active ingredients.

This kit leverages a unique Home compounding approach. In another embodiment of the present invention is a lab bench and retail pharmacy scale compounding kit suitable for customizing a tailor made prescription to fit individual requirements in a dosage form that insures efficacy, swallowability, safety and compliance.

The invention relates to an easy to swallow pharmaceutical composition for oral administration comprising at least one pharmaceutically active compound in an effective amount and comprising a gel agent.

In another embodiment of the present invention, the mixed composition of the present invention may be in a solid form, a gel form or a liquid form.

The present invention proposes an improvement over the art by providing a substantially water free dosage form, containing particulate materials which are further designed for the purpose of masking the taste of drug substances and/or to provide controlled release of a drug substance or drug substances

The present invention further provides a pharmaceutical composition for improving palatability of medicaments. The invention may further provides a taste masking formulation comprising a blend of a lipid(s) in combination with an acid soluble or swellable polymer, which enables delivery of substantial amount of drug immediately, while ensuring improved palatability. Depending on the type of dosage form, the amount of polymer required for imparting palatability will vary.

Palatability of the active pharmaceutical ingredient is a very significant technical obstacle to develop a patient friendly formulation. Organoleptic properties such as taste, mouth feel, smell and the like are considerable factors in distinguishing different products and medicament for same active pharmaceutical index. Therefore the medicament palatability is influenced by a combination of sensory perceptions including taste and smell, and to a lesser extent texture, appearance, and temperature of the product undesirable taste of most drugs is one of the important factors to affect the patient compliance. Furthermore, in the case of pediatric patient for example the taste of the drug product is one f the important criteria when designing the product. Therefore taste masking of bitter drugs is required in order to improve the patient's acceptance and adherence to the particular drug therapy. Various techniques known in the field may be adapted for concealing the objectionable taste of the drug.

The present invention improves the medicament palatability by optimizing these factors. Since there is often a correlation between the chemical structure of a compound and its taste, the present invention provides an optimized composition which is adjusted to the chemical structure and dosage form of the medicament.

The present invention presents a novel technology using geling agent consisting of gellan gum, pregellatinized starch or modified Starch as a carrier and water for a pharmaceutical drug or the like whilst protecting the drug and its function. The gellan gum, pregellatinized starch or modified Starch is in a pH range of about 1-7.5.

Furthermore the present invention provides a composition, a kit and a method configured for converting a prescribed drug into a swallowable drug bolus by using a carrier gel consisting of gellan gum, pregellatinized starch or modified starch for an orally administrated medicine. The carrier is adjusted to the dosage form and amount of the medicament and further to protect the active ingredient of the medicament especially when digestion occurs. The carrier is further adjusted to the dosage, aliquot, tablet, caplet, capsule, pill, bolus, particle, micronized particle, particulate, pellet, core, powder, granule, granulate, small mass, seed, specks, spheres, crystals, beads, agglomerates, nanoparticles of a specific medicament to prevent contamination, overdosing and non sanctioned abuse of active ingredients.

The carrier is designed to hold the medicament together such that it acts as an ‘enteric coating’ or a ‘gastro-resistant’ of the active ingredient within the digestive system without adding any liquid or any other substrate. Further more the composition of the present invention provides a better swallowing form, better smell and taste of the medicament it comprises. Such a composition can be swallowed by even aged people having a weakened swallowing function without causing the improper suction in the trachea.

The gel composition can be easily prepared from a dry gel composition containing a gelling agent capable of rapidly forming a gel upon mixing with water at an ambient temperature in a range of about 20 to 30° C. The gel composition can be formed without the need of heating or cooling the composition.

The composition comprising the gellan gum carrier may further comprise an effective amount of a plasticizer, a disintegration aid, and optionally an effective amount of a slip enhancer.

The plasticizer is selected from the group consisting of propylene glycol, polyethylene glycol 400, polyethylene glycol 3350, polyethylene glycol 8000, glycerol triacetate, polysorbate 80, triethyl citrate, PLAS 2, and acetylated monoglycerides. The disintegration aid is selected from the group consisting of lecithin, pregelatinized modified corn starch, pregellatinized starch, glyceryl carpylate, sorbitan oleate, and sodium lauryl sulfate.

The gellan gum may further comprise a color or colorants or color systems for application to a colored or non colored coated or uncoated dosage form. Illustrative colors and colorants useful herein include without limitation, pigments, dyes, lakes, and oxides (including titanium dioxide) and the like, may be optionally employed with gellan gum used in practicing this invention.

The composition may further comprise binders selected from the group consisting of: hydroxypropyl cellulose, hydroxypropyl methylcellulose and polyvinylpyrrolidone.

In addition, various other ingredients may be employed in the gellan gum composition including any ingredient which is compatible or can be made compatible with the gellan gum composition useful to be mixed with dosage forms presented in this invention. Such other ingredients include, but not limited to, flavor(s), sweetener(s), mint(s), fragrance(s), active ingredient(s) and mixtures thereof and the like. Suitable sweeteners include, but are not limited to, corn syrup solids, sucrose, aspartame, neotame, maltilol, maltilol syrup, neosorb, xylitol, acesulfame, Sweet Am, fructose, brown sugar, Stevia (Butterfly Brand & Wisdom of the Ancients.) Flavors include, but are not limited to, cherry, mint, spearmint, peppermint, wintergreen, banana, coconut, wild cherry, grape, tooti fruiti, cinnamon, strawberry, orange, root beer, bubble gum, chocolate, saccharin and any combination thereof. The gellan gum may be further used as a primary coating, a secondary coating, or a tertiary coating. The gellan gum composition may be coated onto dosage forms which are uncoated or have been coated with one or more prior coatings (overcoating) of an acceptable coating composition which allows adherency with gellan gum. For example, an initial coating may comprise one or more polymers consisting of: cellulosics, dextrins, acrylics, colors, or other pharmaceutical coating material.

Typically this amount of gellan gum is that amount which is necessary to provide an effective or desired coating. The amount of gellan gum which may be added to a dosage form such as tablets in practicing this invention is that amount which provides a gellan gum having a weight gain from about 0.025% to about 30% weight percent of the total medicament weight and preferably from about 0.05% to about 5% weight percent of the total tablet weight although larger and smaller weight percents may be employed if desired

In another embodiment of the present invention, other gelling agents may be used in the present invention such as, Pionil 1500 (a product of Matsutani Chemical Industries Co., Ltd.) is used as the sodium starch phosphate, GENU GEL SWG-J (a product of Copenhagen Pectin Factory) is used as the carrageenin; Primojel (a product of Matsutani Chemical Industries Co., Ltd.) is used as the carboxymethylated starch; GENU pectin is used as the LM pectin, guaiacol is used as the guar gum; L-HPC(LH-31) and L-HPC(LH-21) are used as the low substituted hydroxypropyl cellulose; and Avicel RC-591NF is used as the mixture of crystalline cellulose and sodium carboxymethyl cellulose.

In another embodiment of the present invention, the composition of the present invention may further comprise other suitable gelling and/or swelling a vehicle and/or compositions selected from the group consisting of: hydrocolloids and hydrogelling agents such as alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, or mixtures thereof.

In another embodiment of the present invention, the composition may further comprise sequestrants for facilitating the dissolution of the gellan gum composition. The sequestrants which may be used in the present invention include trisodium orthophosphate (TSP), ethylenediaminetetraacetic acid (EDTA), sodium citrate, tetrasodium pyrophosphate (TSPP), sodium hexametaphosphate (Calgon) and the like. The sequestrants can be added to the aqueous media before or after introduction of the gum. Alternatively, they can be added concomitantly, for example, by preparing dry blends of gum and sequestrant. The amount of sequestrant required to completely dissolve the gum at ambient temperature is a function of the amount of gum, the number of free polyvalent cations in solution, and the particular sequestrant used. The amount of gellan gum which can be used for forming gels ranges from about 0.05-3% (wt./wt.). For many applications levels of 0.05-1% (wt./wt.) are recommended. The gums can be used singly or in combination depending on the properties of the gel which are desired. In another embodiment of the present invention, the composition may further comprise a salt selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations.

In another embodiment of the present invention, the composition can be prepared in a short time in a simplified manner just before the use.

The present invention further provides a method for preparing a composition for oral administration of a medicament, comprising the steps of: (a) providing a kit for oral administration of a medicament comprising: (i) a container for mixing a crushed medicament with a carrier comprising a lower chamber and an upper chamber connected with each other and a membrane for separating the chambers, (ii) at least one additive, (iii) a crushing apparatus for crushing a medicament, (iv) a collecting device, (v) a medicament and, (vi) a carrier consisting of gellan gum, pregellatinized starch or modified starch.

(b) crushing the medicament using a crushing apparatus, (c) adjusting the upper chamber to the dosage form of the medicament, (d) applying the gel-based carrier and the medicament to the upper chamber of the container whilst the lower chamber comprises water and salt solution, (e) removing or partially breaking the membrane of the container; and

(f) admixing the lower and upper chambers comprising gellan gum and water under shear to prepare a gellan gum composition.

The composition of the present invention may be in a solid form, a gel form or a liquid form.

In another embodiment of the invention, the method may comprise the step of applying the gel-based carrier to the first chamber of the container whilst the second chamber comprises water and salt solution. The medicament and additional additives may be applied to the first chamber together with the carrier or optionally to the second chamber together with the water.

In another embodiment of the invention the method as described above, wherein the gel-based carrier is adapted as an enteric coating of the medicament; In another embodiment of the invention the method as described above, wherein the enteric coating is adjusted to the dosage form of the medicament in order to prevent contamination, overdosing and non sanctioned abuse of active ingredients.

In another embodiment of the invention the method as described above, wherein the dosage form selected from the group consisting of: aliquot, tablet, caplet, capsule, pill, bolus, particle, micronized particle, particulate, pellet, core, powder, granule, granulate, small mass, seed, specks, spheres, crystals, beads, agglomerates, nanoparticles or any combination thereof.

In another embodiment of the invention the method as described above, wherein further comprises an effective amount of a plasticizer, a disintegration aid, or an effective amount of a slip enhancer.

In another embodiment of the invention the method as described above, wherein the gellan gum has a weight of about 0.025% to about 30% weight percent of the total medicament dosage form weight.

In another embodiment of the invention the method as described above, wherein the gellan gum has a weight of about 0.025% to about 10% weight percent of the total medicament dosage form weight.

In another embodiment of the invention the method as described above, wherein the gellan gum has a weight of preferably from about 0.05% to about 5% weight percent of the total dosage form weight.

### Example 1

### Starch Gel Preparation from a Dosage Form without Protective Coating and without Sustained/Extended Release Matrix, Using the Following

Pregellatinized starch (800 mg), Maltodextrine (300 mg), Sweetener (Sucrazit) 80 mg, Strawberry flavoring, Pink coloring, Dexamol (Paracetamol) 500 mg, Purified water (10 ml).

The tablet was crushed using a crushed apparatus and added to the upper chamber of the container at a room temperature and standard conditions. After admixing by rotating and shaking the upper chamber at least half-turn (relatively to the lower chamber) the membrane (separating the upper and lower chamber) is broken forming an aperture in the middle of the membrane. The apparatus is of a semi rigid material such that when tapping, knocking, raping or just hitting the upper chamber the membrane will brake in the middle forming an aperture. The formed aperture allows the content of the upper chamber to be transferred to the lower chamber. The apparatus comprising the mixture was mixed again forming a homogeneous medicament composition. The prepared medicament composition was extruded by removing the upper chamber. The prepared medicament composition had a taste with pudding consistency and was further easy to swallow.

FIG. 5 further presents a release profile graph of acetaminophen (Paracetamol) as a blank measurement.

A tablet of Acetaminophen (Dexamol, 500 mg, Dexcel Pharma) was grinded to fine powder using mortar and pestle. The powder was introduced to a beaker with 100 ml of simulated gastric fluid (SGF, 37° C., pH 1.2) and were mixed slowly and gently at least 6 rounds per minute, using magnetic stirrer. 100 μL of the mixture was sampled and methanol was added up to whole content of 1 ml. The prepared sample was filtered and used for absorbance measurement (Cole-Parmer Scanning Double Beam UV/Visible Spectrophotometer). An absorbance of the prepared sample was observed at 243.5 nm.

FIG. 6 further illustrates a release profile graph of acetaminophen admixed with starch gel. The gel prepared as described in Example 1 was used. The measurement technique and dissolution medium type were identical to those of the blank measurement. An absorbance was observed at 243.5 nm.

The results show that after 25 minutes in the SGF, a 100% of acetaminophen was released to the medium, as compared with the blank measurement, thus, indicating the same release profile comparing to an in vivo medicament release profile in a patient stomach. (The In vitro drug dissolution data generated from dissolution testing experiments was further related to in vivo pharmacokinetic data by means of in vitro-in vivo correlations (IVIVC)).

### Example 2

### Starch Gel Preparation from a Dosage Form without Protective Coating and without Sustained/Extended Release Matrix, Using the Following

Modified starch (acetylated distarch phosphate) 800 mg, Maltodextrine 500 mg Sweetener (Sucrazit) 82 mg, Strawberry flavoring, Pink coloring, CaCl₂, Dexamol (Paracetamol) 500 mg, Purified water (10 ml).

The procedure of the gel preparation is identical to this of the Example 1. The prepared gel was tasty and easy to swallow.

### Example 3

### Starch Gel Preparation from a Dosage Form without Protective Coating and without Sustained/Extended Release Matrix, Using the Following

Pregellatinized starch (800 mg), Sugar (400 mg), Orange flavoring, Orange coloring CaCl₂, Dexamol (Paracetamol) (500 mg), Purified water (10 ml).

The procedure of the gel preparation is identical to this of the Example 1. The prepared gel was tasty and easy for swallowing.

### Example 4

### Gellan Gum Gel Preparation from a Dosage Form with Protective Coating or with Sustained/Extended Release Matrix, Using the Follow

Gellan Gum (50 mg), Pregellatinized starch (200 mg), Sugar (300 mg) Orange flavoring, Orange coloring, Dexamol (Paracetamol) 500 mg, Deionized water (10 ml).

The procedure of the gel preparation is identical to Example 1 except than the nature of the gellation agent.

FIG. 7 further illustrates the release profile of acetaminophen admixed with Gellan Gum gel as described in Example 4. The measurement technique was identical to the Blank measurement. An absorbance was observed at 243.5 nm.

After the preparation, the gel that contains the medicine was introduced to a beaker with 100 mL SGF equipped with magnetic stirrer. The gel particles immediately changed their texture to more solid and hard in this medium. After 120 min, a monobasic potassium phosphate (0.68 g) was added and the pH was adjusted to 6.8 by addition of 0.2 N sodium hydroxide (simulated intestinal fluid (SIF) sine pancreatin). The gel particles changed their texture with a time to more soft. Each 5 min a 100 μL of the fluid were sampled and a methanol was added up to whole volume of 1 mL. Then, the sample was filtered and an absorbance value was observed at 243.5 nm After 1 h of the measurement, the fluid was sampled each 15 min.

The results show very low release rate within 120 min (up to 6% in the SGF). After the 120 min interval (SIF), a gradual elevation of the acetaminophen release occurs. During 7.5 hours of the test, the release level reached 78%, as compared with the blank measurement. Thus, a medicine gets a protection in the stomach by the formation of a more hard gel and by critically decreased release of the medicine. After a passing along the intestine, the pH is increase and the gel swelling and releases the medicine. The release rate depends on the gel composition, the amount of the gel agents, the medicine nature, the temperature, and the pH.

### Example 5

### Gellan Gum Gel Preparation from a Dosage Form with Protective Coating or with Sustained/Extended Release Matrix

Gellan Gum (100 mg), CaSO₄ (25 mg), Sodium citrate (21 mg), Sugar (300 mg) Strawberry flavoring, Pink coloring, Dexamol (Paracetamol) 500 mg, Deionized water (10 mL).

The procedure of the gel preparation is identical to this of the Example 4.

Pregellatinized Starch Gel Dissolution Assay;

Each sample was prepared from pregellatinized starch (800 mg), maltodextrin (300 mg), and distilled water (10 mL). 30 sec after the preparation, each sample was placed to vial comprising aqueous media with specific pH value. A pH values were achieved by addition of HCl/NaOH to purified water and measured using a pH meter.

TABLE 1

| | | time to gel | |
|---|---|---|---|
| pH | starch | breakdown | observation |
| 1 | 1, 2 | 8 min | partial transparent colorless gel dissolves |
| 2 | 1, 2 | 8 min | partial transparent colorless gel dissolves |
| 3 | 1, 2 | 10 min | partial transparent colorless gel dissolves |
| 4 | 1, 2 | 10 min | partial transparent colorless gel dissolves |
| 5 | 1, 2 | 11 min | partial transparent colorless gel dissolves |
| 6 | 1, 2 | 11 min | partial transparent colorless gel dissolves |
| 7 | 1, 2 | 11 min | partial transparent colorless gel dissolves |

Legend:
starch 1 - Emgel EP-300 EMSLAND STAERK GMBH
starch 2 - Emgel EP-820C EMSLAND STAERK GMBH

The dissolution was determined by visual test of each vial and by filtration of each sample via filtration paper and examining the solids.

In the aqueous media of all the pH values a time of gel dissolution was 8-11 min. So, after swallowing of the gel, it's structure destroyed within this time interval and, thus, it can't influence a medicine release.

Reference is now made to FIGS. 8 and 9 which illustrates the device of the present invention for compounding an orally administered dosage form of at least one medicament, comprising: a container 400 for mixing a crushed medicament with a gel-based carrier. The container comprising at least one first chamber 410 for accommodating a solution and at least one second chamber 420 for accommodating a gel-based carrier. The first chamber and the second chamber are connected with each other via a membrane which further used as a separating element between the first and the second chamber. The container further comprises a cap 400a for inserting a medication and solution into the first chamber.

The device and more particularly the membrane is composed of a flexible, semi rigid material selected from the group consisting of Acrylic and modified acrylic plastics, Acrylonitrile/butadiene/styrene co-polymer, Acrylonitrile/butadiene/styrene/methyl methacrylate copolymer, Acrylonitrile/styrene copolymer, n-Alkylglutarimide/acrylic copolymers, Cellophane, Cross-linked polyacrylate copolymers, 1,4-Cyclohexylene dimethylene terephthalate, 1,4-cyclohexylene dimethylene isophthalate copolymer, Ethylene-acrylic acid copolymers, Ethylene-carbon monoxide copolymers, Ethylene-1, 4-cyclohexylene dimethylene terephthalate copolymers, Ethylene-ethyl acrylate copolymers, Ethylene-methyl acrylate copolymer resins, Ethylene/1,3-phenylene oxyethylene isophthalate/terephthalate copolymer, Ethylene-vinyl acetate copolymers, Ethylene-vinyl acetate-vinyl alcohol copolymers, Fluorocarbon resins, Hydroxyethyl cellulose film, water-insoluble, Isobutylene polymers, Isobutylene-butene copolymers, Nylon resins, Olefin polymers, Perfluorocarbon resins, Polyarylate resins, Polyaryletherketone resins, Polyarylsulfone resins, Poly-1-butene resins and butene/ethylene copolymers, Polycarbonate resins, Polyestercarbonate resins, Polyester elastomers, Polyetherimide resin, Polyethylene resins, carboxyl modified, Polyethylene, chlorinated, Polyethylene, fluorinated, Polyethylene, oxidized, Polyethylene phthalate polymers, Poly(phenyleneterephthalamide) resins, Poly(p-methylstyrene) and rubber-modified poly(p-methylstyrene), Poly(oxy-1,2-ethanediyloxycarbonyl-2,6-naphthalenediylcarbonyl) resins, Polystyrene and rubber-modified polystyrene, Polysulfide polymer-polyepoxy resins, Polysulfone resins, Poly (tetramethylene terephthalate), Polyvinyl alcohol film, Polyurethane resins, Styrene block polymers, Styrene-maleic anhydride copolymers, Styrene-methyl methacrylate copolymers, Textryls, Urea-formaldehyde resins in molded articles, Melamine-formaldehyde resins in molded articles, Vinyl chloride-ethylene copolymers, Vinyl chloride-hexene-1 copolymers, Vinyl chloride-lauryl vinyl ether copolymers, Vinyl chloride-propylene copolymers, Vinylidene chloride/methyl acrylate copolymers, Vinylidene chloride/methyl acrylate/methyl methacrylate polymers and any combination thereof.

FIG. 9 further illustrates the second chamber structure 430 for contacting the gel-based carrier fraction.

In another embodiment of the present invention, the membrane comprising a reversibly sealed orifice or aperture such that when opened, provides a mixture of a defined dosage form of the medicament comprising the crushed medicament and the gel-based carrier. The container further comprises a disposable cap for extracting the prepared mixture.

Reference is now made to FIGS. 10 and 11 which illustrate a cross section of the device of the present invention comprising the inner membrane 540. As illustrated in FIG. 10 the first chamber comprises a cap 510 for inserting the solution and a membrane in between the first 560 and second chamber 520. The chambers of the device may be detachable and replaceable portions. FIG. 11 further illustrates the membrane 540 comprising the reversibly sealed orifice which may be opened and closed automatically or manually. The device may further comprise a sensor controlling the open and/or closed configuration of the membrane's orifice.

In another embodiment of the present invention, a method for preparing a composition for oral administration of at least one medicament, comprising the steps of: opening the cap, transfer crushed medicine to first chamber comprising a solution such as water, closing the cap, shaking or rotating the device for mixing the crushed medicine with the water, rotating, pushing, pulling or knocking at least one chamber thereby forming an orifice within the membrane such that the mixed solution comprising the medicine is transferred to the lower chamber. The following step is rotating at opposite direction the first chamber such that the orifice of the membrane is hermetically closed. The device is than shook for a number of seconds forming a gel formation. In order to extract the formed gel the cap is disposed by pressing the first chamber to take out the formed tasty gel using a manually operated cover.

Reference is now made to FIGS. 12 and 13 which illustrate a device for compounding an orally administered dosage form of at least one medicament, comprising: a container 600 for mixing a crushed medicament with a gel-based carrier. The container comprising at least one first chamber 610 for accommodating a solution and at least one second chamber 620 for accommodating a gel-based carrier. The first chamber and the second chamber are connected with each other via a membrane 630 having at least one reversible orifice which further used as a separating element between the first and the second chamber. At least one of the chamber may comprise a cover which may be a disposable cover 660 or interconnected to the edge of the chamber 670.

In another embodiment of the present invention, the chambers may be configured as a piston-like such that the first chamber can be inserted within the second chamber.

In another embodiment of the present invention, at least one of the chambers may further comprise excipients (fillers) such as bulking agents or diluents selected from the group consisting of lactose, sucrose, glucose, mannitol, sorbitol, magnesium stearate, plant cellulose, calcium carbonate, calcium phosphate, ascorbic acid and any mixture thereof. The fillers are added to prevent the formation of agglomerates and means thereof. The second chamber comprising the gel-base carrier further includes a cover which can be removed when a mixture is prepared and further for administrating the mixture.

In order to extract the formed gel the cap is disposed by pressing the upper side of the first chamber to take out the formed tasty gel using a piston-like action.

Reference is now made to FIGS. 14 and 15 which illustrate a cross section of the device of the present invention 700 comprising a membrane 710 in between the chambers and a cap 720. As illustrated in FIG. 15 the membrane 730 located between the chamber comprises an orifice allowing the content of one chamber to be transferred and further mixed within the second chamber. The device may be based upon a syringe-like mechanism such that at least one chamber is configured as a plunger which can be pulled and pushed along inside a second chamber configured as a cylindrical tube. The syringe-like configuration allows at least one chamber to rotate, spin, to turn around or to be ejected from the second chamber. The syringe-like configuration further provides degrees of freedom of at least one chamber such that at least one chamber may further at least partially move or rotate in an horizontal or vertical direction. At least one chamber can be screwed into a second chamber.

In another embodiment of the present invention, the method for preparing a composition for oral administration of at least one medicament, comprising the steps of: providing a device for oral administration of a medicament comprising: a container for mixing a crushed medicament with a gel-based carrier comprising at least one first chamber and at least one second chamber connected with each other via a membrane for separating the chambers. The membrane comprising a reversibly sealed orifice, (i) a crushed medicament; and (ii) a gel-based carrier.

The method further comprises the following steps applying a gel-based carrier and a medicament to the first chamber of the container whilst the second chamber comprises water,

opening the membrane's orifice by pressing an operation button or by rotating, spinning, turning or twisting the first chamber at least a half-turn therefore, and admixing the constant of the first chamber and the second chamber forming a gel-like mixture comprising the gel-based carrier, medicament and water under shear to prepare a solid composition.

Reference in now made to FIGS. 16-18 which illustrate the upper view of the distal and proximal portions of the device of the present invention. FIG. 16 illustrates the distal portion of the device comprising a chamber comprising water and medication and further a cover 800 for inserting substances and further closing the chamber. FIG. 17 further illustrates the membrane portion 810 comprising at least one orifice which can be opened and closed following a manual action such as spinning at least one chamber at least a portion of a turn or circle. A portion of a turn is selected from a group consisting of: at least a quarter-turn, at least a half-turn and any combination thereof. FIG. 18 further illustrates the upper view of the proximal portion of the device comprising a chamber with a disposable cover 820 after extracting the prepared pharmaceutical composition.

Reference is now made to FIGS. 19 and 20 which illustrates an HPLC chromatography assay and dissolution of paracetamol in paracetamol gel, 500 mg/dose for identification, content, quantify and dissolution of the prepared composition comprising the gel-based carrier, a solution and a crushed medication. The active ingredient of the crushed medication which was used is paracetamol. FIG. 11 illustrates an HPLC standard area and the retention time of paracetamol 500 mg.

The sample mixture to be separated and analyzed is introduced, in a discrete small volume (typically microliters), into the stream of mobile phase percolating through the column. The components of the sample move through the column at different velocities, which are function of specific physical interactions with the sorbent (also called stationary phase). The velocity of each component depends on its chemical nature, on the nature of the stationary phase (column) and on the composition of the mobile phase. The time at which a specific analyte elutes (emerges from the column) thereby its retention time was measured under particular conditions and is considered an identifying characteristic of a given analyte.

The HPLC procedure and sample preparation included 5 replicate injections of first standard solution and additional 2 injections of a second standard solution were identified and analyzed. The release of paracetamol in mg dose of pharscetamol gel, 500 mg during dissolution test after 30 min, was calculated according to the following equation:

$$Content = \frac{{W}_{st} \times P \times {A}_{s} \times 10}{{A}_{st}} , where \\ {W}_{st} - Weight of standard in mg , \\ {A}_{st} - \begin{matrix}area of Paracetamol peak in \\ standard chromatogram\end{matrix} , \\ {A}_{s} - area of Paracetamol in sample chromatogram , \\ P - Purity of Paracetamol standard , a . u . \\ 10 - dilution factor . \qquad Equation 1$$

The retention time (RT) of paracetamil is 7.5+_1.0 min

FIG. 20 further illustrates the release of paracetamol in % L.C units from paracetamol gel, 500 mg/dose according to equation 2, as follows,

$$\% Release = \frac{{W}_{st} \times P \% \times {A}_{s} \times 9}{{A}_{st}} , where \\ {W}_{st} - Weight of standard in mg , \\ {A}_{st} - area of Paracetamol peak in \\ standard chromatogram , \\ {A}_{s} - area of Paracetamol in sample chromatogram , \\ P \% - Purity of Paracetamol standard , in \% \\ 500 - Label Claim of Paracetamol \\ in Paracetamol Tablet , 500 mg \\ 9 - dilution factor . \qquad Equation 2$$

As illustrated, the paracetamol retention time was detected after 7.79 minutes similar to the standard retention time.

### Example 5

A gel-based carrier was prepared to serve as a vehicle for oral dosage forms administration. The initial product may be a mix of dry powders containing crushed medication, turning to gel after addition of water. The gel-based carrier is characterized by gelatinization, homogeneity formulation and further API release similar as the paracetamol medication.

The gel which may be used are dispersed and neutralized carbomer-based or long dispersion and mixed cellulose-based gel.

A 100% of pregelatinized starch was used. An additional dry water soluble excipients (filler) such as sugar, ascorbic acid and sodium saccharin were used.

In 20 ml scintillation vial, ascorbic acid, sodium saccharin, pregelatinized starch and crushed Acamol tablet have been mixed. The powders were added to 10 ml of water and after shaking for a few seconds. the gel containing a crushed tablet was obtained.

Another gel formulation without any clump eas based on gum guar. Gum guar forms gels during a few seconds. The quantity of the gum guar was optimized to achieve the limits of dissolution test (see table 1)

The FDA requirements of paracetamol tablet is >85% release for 30 min both formulations are based on gum guar and on pregelatinized starch.

The release of the paracetamol from the developed gel containing crushed medication is equivalent to the release of the paracetamol from the medication

TABLE 2
Table 1. Release of Paracetamol from different vehicles

| Lot | Description | Release after 30 min, % |
|---|---|---|
| 030414 | 3% gum guar | 52 |
| 37 | 2% gum guar | 85 |
| 39 | 6.7% starch | 91 |
| Acamol Tablet, 500 mg | | 88 |

### Example 6

Ingredients: water, sweetener, colorant, odor and taste additives. Volume of the syrup inside a bottle is 10-30 mL.

After opening of a bottle with the syrup, a medicine powder must be inserted into the bottle, and after shaking of the mixture the tasty syrup with the medicine can be eaten.

### Example 7

Ingredients of a ready gel: water, gelating agent (Gelan Gum, pregellatinized starch, modified starch or other gelating agent), sweetener, colorant, odor and taste additives. The gel was prepared before a use.

The gel on basis of Gelan Gum, pregellatinized starch, modified starch or other gelating agents is ready to eating and intended for mixing with crushed medicine. The bottle (300-2000 mL) equipped with dispenser and contains gel of jelly/pudding texture. After a pushing of the dispenser, the gel may be collected into a small cup and mixed with crushed medicine. After the mixing, the tasty gel can be eaten.

1-59. (canceled)

60. An apparatus for compounding an orally administered dosage form of at least one medicament comprising: a container for mixing a crushed medicament with a gel-based carrier; said container comprises at least one chamber and at least one membrane as a separating element;
wherein said membrane, when removed, provides a mixture of a defined dosage form of said medicament comprising said crushed medicament and said gel-based carrier; said membrane is further configured to prevent contamination, overdosing and non sanctioned abuse of active ingredient of said medicament.

61. The apparatus according to claim 60, wherein said container comprising at least one first chamber for accommodating a solution and at least one second chamber for accommodating a gel-based carrier; said at least one first chamber and at least one second chamber are connected with each other via a membrane for separating said first and second chambers; said membrane, when removed, provides a mixture of a defined dosage form of said medicament comprising said crushed medicament and said gel-based carrier, said first chamber and second chamber have a surface tension allowing the user to extract said mixture by pressing at least one chamber.

62. The apparatus according to claim 60, wherein at least one of the following hold true:
a. said membrane comprising a reversibly sealed orifice such that when opened, provides a mixture of a defined dosage form of said medicament comprising said crushed medicament and said gel-based carrier; said reversibly sealed orifice is opened by manual action or a manual or electronic button;
b. said membrane is partially broken by rotating said first chamber at least a half-turn therefore, providing a defined dosage form of said medicament comprising said crushed medicament and said gel-based carrier, said membrane is further configured to prevent contamination, overdosing and non sanctioned abuse of active ingredient of said medicament;
c. additionally comprising a collecting device selected from the group consisting of a spoon, a cup and a combination thereof; and
d. additionally comprising a crushing apparatus for crushing a medicament.

63. The apparatus according to claim 60, wherein at least one of the following holds true:
a. said membrane is a composed of a flexible material selected from the group consisting of polymer, cellulose, metal, metal alloys, metal oxides and any combination thereof;
b. said membrane is partially broken by tapping, knocking, raping or hitting the upper chamber thereby forming an aperture within a portion of the membrane;
c. said dosage form selected from the group consisting of: aliquot, tablet, caplet, capsule, pill, bolus, particle, micronized particle, particulate, pellet, core, powder, granule, granulate, small mass, seed, specks, spheres, crystals, beads, agglomerates, nanoparticles or any combination thereof;
d. said at least one chamber is an upper chamber comprising at least one mold adjusted to a structure of said dosage form of at least one medicament; and
e. said at least one first chamber is adapted for accommodating said gel-based carrier whilst said second chamber is adapted for accommodating water and salt;

64. The apparatus according to claim 60, wherein at least one of the following holds true:
a. said salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
b. said container is suitable for customizing a tailor made prescription to fit individual requirements in a dosage form that insures efficacy, swallowability, safety and compliance;
c. said mold has a cavity shape selected to accommodate a dosage form selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; said mold is a replaceable element which can be removed and/or replaced;
d. said gel-based carrier is selected from the group consisting of gellen gum, pregellatinized starch, modified starch, Pionil 1500, GENU GEL SWG-J, Primojel, GENU pectin, guaiacol, L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof; and
e. said container additionally comprising a syringe or a needle attached to at least one edge of said container for dispensing said mixture.

65. A kit for oral administration of at least one medicament comprising:
a. an apparatus for compounding an orally administered dosage form of a medicament comprising:
i. a container for mixing a crushed medicament with a carrier said container comprising at least first chamber and at least second chamber connected with each other via a membrane for separating said chambers; and,
ii. a collecting device;
b. a crushing apparatus for crushing a medicament;
c. a medicament; and,
d. a gel-based carrier selected from the group consisting of gellan gum, pregellatinized starch, modified starch and a combination thereof;
wherein said membrane is removed or partially broken by rotating said first chamber at least a half-turn therefore, providing a mixture of defined dosage form of a defined medicament comprising said crushed medicament and said gel-based carrier, so as to prevent contamination, overdosing and non sanctioned abuse of active ingredient of said medicament.

66. The kit according to claim 65, wherein said membrane, when removed, the content of each of said chamber is mixed thereby, forming a mixture of a defined dosage form of a defined medicament comprising said crushed medicament and said gel-based; said first chamber and second chamber have a surface tension allowing the user to extract said mixture by pressing at least one chamber.

67. The kit according to claim 65, wherein at least one of the following holds true:
a. said dosage form is selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof;
b. said at least one chamber comprising at least one mold adjusted to a structure of said dosage form of at least one medicament;
c. said membrane is partially broken by tapping, knocking, raping or hitting said first chamber thereby forming an aperture within a portion of said membrane;
d. said mold has a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; said mold is a replaceable element which can be removed and/or replaced; and
e. said collecting device is selected from the group consisting of spoon, cup and any combination thereof.

68. The kit according to claim 65, wherein least one of the following holds true:
a. said at least one chamber is adjusted to the dosage form of said medicament preventing contamination, overdosing and non sanctioned abuse of active ingredients of said medicament;
b. said kit is suitable for customizing a tailor made prescription to fit individual requirements in a dosage form that insures efficacy, swallowability, safety and compliance;
c. said second chamber is adapted for accommodating water and salt;
d. said salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations; and
e. said gel-based carrier is further selected from the group consisting of Pionil 1500, GENU GEL SWG-J, Primojel, GENU pectin, guaiacol, L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof.

69. A solid dosage form composition for oral administration prepared by a process comprising steps of:
a. providing a gel-based carrier adapted for mixing with orally administrated medicament; said gel-based carrier is selected from the group consisting of: gellan gum, pregellatinized starch, modified starch and a combination thereof;
b. providing a crushed or powdered dosage form of a medicament;
c. providing an apparatus for compounding an orally administered dosage form of a medicament comprising:
i. a container for mixing a crushed medicament with a gel-based carrier; said container comprising at least one first chamber and at least one second chamber connected with each other via a membrane for separating said first chamber and said second chamber;
ii. a crushing apparatus for crushing a medicament; and,
iii. a collecting device;
d. admixing the content of said first chamber comprising said carrier and said second chamber comprising water under shear to prepare a solid composition; wherein said step of admixing the content is by removing said membrane from said container thereby, providing a mixture of a defined dosage form of said medicament comprising said crushed medicament and said gel-based carrier; further wherein said first chamber and second chamber have a surface tension allowing the user to extract said mixture by pressing at least one chamber.

70. The composition according to claim 69, wherein said step of admixing the content is by additionally rotating said first chamber at least a half-turn membrane such that said membrane is partially broken therefore, providing a mixture of defined dosage form of a defined medicament comprising said crushed medicament and said gel-based carrier, so as to prevent contamination, overdosing and non sanctioned abuse of active ingredient of said medicament.

71. The composition according to claim 69, wherein at least one of the following holds true:
a. said solid composition is useful for improving swallowing form, smell and taste of the medicament it comprises; and
b. said membrane is partially broken by tapping, knocking, raping or hitting at least one chamber thereby forming an aperture within a portion of said membrane.

72. The composition according to claim 69, wherein at least one of the following holds true:
a. further comprising additives as a palatability improving agents;
b. said solid composition further comprising an effective amount of a plasticizer, a disintegration aid, or an effective amount of a slip enhancer;
c. said composition further comprises binders selected from the group consisting of: hydroxypropyl cellulose, hydroxypropyl methylcellulose and polyvinylpyrrolidone; and
d. said composition further comprises ingredients selected from the group consisting of: flavor(s), sweetener(s), mint(s), fragrance(s), active ingredient(s) and mixtures thereof.

73. The composition according to claim 69, wherein at least one of the following holds true:
a. said dosage form is selected from the group consisting of: aliquot, tablet, caplet, capsule, pill, bolus, particle, micronized particle, particulate, pellet, core, powder, granule, granulate, small mass, seed, specks, spheres, crystals, beads, agglomerates, nanoparticles or any combination thereof;
b. said at least one chamber comprising at least one mold adjusted to a structure of said dosage form of at least one medicament; said mold is with a cavity shape to accommodate a dosage form selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; said mold is a replaceable element which can be removed and/or replaced; and
c. said collecting device is selected from the group consisting of: spoon, cup or the like.

74. The composition according to claim 69, wherein at least one of the following holds true:
a. said gellan gum is provided in a weight of about 0.025% to about 30% weight percent of the total dosage form weight; and
b. said gellan gum is provided in a weight of preferably about 0.05% to about 5% weight percent of the total dosage form weight.

75. The composition according to claim 69, wherein at least one of the following holds true:
a. said at least one chamber comprising water with a salt selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
b. said sweeteners is selected from the group consisting of corn syrup solids, sucrose, aspartame, neotame, maltilol, maltilol syrup, neosorb, xylitol, acesulfame, Sweet Am, fructose, brown sugar, Stevia, saccharin and a combination thereof;
c. said flavors is selected from the group consisting of cherry, mint, spearmint, peppermint, wintergreen, banana, coconut, wild cherry, grape, tooti fruiti, cinnamon, strawberry, orange, root beer, bubble gum, chocolate and any combination thereof; and
d. said gel-based carrier is selected from the group consisting of Pionil 1500, GENU GEL SWG-J; Primojel, GENU pectin, guaiacol; L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof.

76. A method for preparing a composition for oral administration of at least one medicament, comprising steps of:
a. providing a kit for oral administration of a medicament comprising:
i. a container for mixing a crushed medicament with a gel-based carrier comprising at least one first chamber and at least one second chamber connected with each other via a membrane for separating said chambers a crushing apparatus for crushing a medicament; and,
ii. a collecting device;
iii. a medicament; and
iv. a gel-based carrier selected from the group consisting of gellan gum, pregellatinized starch, modified starch and a combination thereof;
b. crushing said medicament using a crushing apparatus;
c. adjusting at least one chamber to the dosage form of said medicament;
d. applying said carrier to said first chamber of said container whilst the second chamber comprises water and salt solution;
e. removing or partially breaking said membrane of said container; and
f. admixing said first chamber and second chamber comprising said gel-based carrier, medicament and water under shear to prepare a solid composition.

77. The method according to claim 76, wherein at least one of the following holds true:
a. said method additionally comprising the step of providing said gellan gum as an enteric coating of said medicament; said enteric coating is adjusted to the dosage form of said medicament in order to prevent contamination, overdosing and non sanctioned abuse of active ingredients of said medicament;
b. said method further comprising the step of adding a salt solution to said chamber comprising water; said salt is selected from the group consisting of: calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, alkali metal chlorides, sodium fluoride, organic acids such s citric, succinic, fumaric, malic, maleic, glutaric, lactic and the like; alkali metal sulfates such as sodium sulfate; dihydrogen sodium phosphate, monohydrogen sodium phosphate, disodium hydrogen phosphate, and mixtures thereof, and multivalent metal cations;
c. said method further comprising the step of adding an effective amount of a plasticizer, a disintegration aid, or an effective amount of a slip enhancer;
d. said method additionally comprising step of providing said gellan gum in a weight of preferably from about 0.05% to about 5% weight percent of the total medicament dosage form weight; and
e. said method additionally comprising step of providing said gellan gum in a weight of about 0.025% to about 30% weight percent of the total medicament dosage form weight.

78. The method according to claim 76, wherein at least one of the following holds true:
a. said step of providing said membrane comprising a reversibly sealed orifice;
b. said method additionally comprising step of opening said membrane's orifice by using a manual action or a manual or electronic button;

79. The method according to claim 76, wherein at least one of the following holds true:
a. said step of breaking partially said membrane is further by tapping, knocking, raping or hitting said first chamber thereby forming an aperture within a portion of said membrane;
b. said step of adjusting said at least one chamber to the dosage form of said medicament using a mold having a cavity shape selected from the group consisting of: tablet, caplet, particle, micronized particle, particulate, pellet, pill, core, powder, granule, granulate, small mass, seed, specks, spheres, crystals, beads, agglomerates, nanoparticles or any combination thereof; and
c. said step of providing said container comprising a syringe or a needle attached to at least one edge of said container for dispensing said mixture.
